(19) United States
(12) Patent Application Publication
(10) Pub. No.: US 2001/0006645 A1
(43) Pub. Date: Jul. 5, 2001

(54) METHOD FOR TRANSDERMAL DRUG DELIVERY

(76) Inventors: JANE A. NORTON, LEXINGTON, KY (US); JAMES A. MCCUBBIN, SENECA, SC (US)

Correspondence Address: KING & SCHICKLI, PLLC, 247 NORTH BROADWAY, LEXINGTON, KY, 40507, US

(21) Appl. No.: 08/956,319

(22) Filed: Oct. 23, 1997

Related U.S. Application Data
(60) Provisional application No. 60/029,645, filed on Oct. 28, 1996.
(60) Provisional application No. 60/049,930, filed on Jun. 18, 1997.

(51) Int. Cl. A61K 9/00

(52) U.S. Cl. 424/400

(57) ABSTRACT

A method for the transdermal delivery of a selected drug, particularly for the purposes of immunization and vaccination, is provided. The method broadly includes the steps of (1) preparing an area of the skin, through which the transdermal delivery is to be made, by using alpha hydroxy acid to exfoliate the skin area; (2) providing a medicated patch which contains the selected drug to be delivered transdermally, the patch also containing propylene glycol for facilitating the transdermal delivery; and (3) applying the patch to the area of skin which was previously prepared using the alpha hydroxy acid.

This application claims the benefit of U.S. Provisional application Ser. No. 60/029,645, filed Oct. 28, 1996 and U.S. Provisional application Ser. No. 60/049,930, filed Jun. 18, 1997.

### TECHNICAL FIELD

The present invention relates generally to drug delivery systems and, more particularly, to a method for the transdermal delivery of a selected drug.

### BACKGROUND OF THE INVENTION

An effective and safe means for drug delivery is essential in maintaining a healthy society. For example, the ability to provide a quick and simple immunization against preventable and infectious diseases can usually deter and prevent the spreading of such diseases. However, despite the advancements of modern medical technology it is too often the case that a quick and simple immunization procedure cannot be provided. In fact, numerous sources indicate that infants, children and the population in general are not being adequately immunized against preventable and infectious diseases. These unprotected individuals are found in all demographic and socioeconomic categories. There has been much public discussion of this public health/medical dilemma and it is apparent that this is a problem on a global scale.

The Pediatric Academy of Children reports an immunization rate of approximately 70%, indicating about 30% are unprotected nationally. This is likely to be a significant underestimation because of the large numbers of unregistered immigrants. In an attempt to solve this problem, the U.S. Department of Health and Human Services on Oct. 1, 1994 implemented the vaccines for children (VFC) program, which provides free vaccine for children at participating private and public health care provider sites. In addition, uninsured children can receive vaccines through the VFC at federally qualified health care centers and rural health clinics. Other children can receive free vaccines at public clinics under existing programs.

Despite these new federal mandates, the existing delivery systems have a number of problems that contribute to the high incidence of vaccine preventable childhood diseases. Availability of existing vaccines is only one of several challenges that must be overcome to achieve vaccination goals. In addition access, delivery and compliance are interwoven barriers that result in under protection of the national and global population.

Conventional means for providing drug delivery, and more particularly for providing immunization, usually includes a form of hypodermic injection. However, there are often problems or complications associated with drug delivery by injection. These problems include, for example, fear and trauma caused by injection, immediate pain, muscle inflammation and the immediate risk of anaphylaxis. Additionally, the safe handling and disposal of used hypodermic needles is a critical problem if the inadvertent spread of diseases such as Acquired Immune Deficiency Syndrome (AIDS) from accidental “sticks” is to be avoided.

The present invention relies upon percutaneous absorption: that is, the transfer of drug from the skin surface into the stratum corneum, under the aegis of a concentration gradient, and its subsequent diffusion through the stratum corneum and the underlying epidermis, through the dermis and into the microcirculation. Thus, drug delivery is achieved without the pain and trauma often associated with hypodermic injection. Further, the difficult task of safely disposing of a used hypodermic needle is avoided since no such needle is used.

### SUMMARY OF THE INVENTION

Accordingly, it is a primary object of the present invention to provide a method for the delivery of a selected drug overcoming the above described limitations and disadvantages of the prior art.

Another object of the present invention is to provide a delivery system and method capable of efficiency delivering a wide range of beneficial drugs/immunizations in a cost effective, relatively painless and convenient manner.

Still another object of this invention is to provide a method for the transdermal delivery of a selected drug exhibiting improved efficacy over conventional means of drug delivery.

Additional objects, advantages and other novel features of the invention will be set forth in part in the description that follows and in part will become apparent to those skilled in the art upon examination of the following or may be learned with the practice of the invention. The objects and advantages of the invention may be realized and obtained by means of the instrumentalities and combinations particularly pointed out in the appended claims.

To achieve the foregoing and other objects, and in accordance with the purposes of the present invention as described herein, a method for the transdermal delivery of a selected drug is provided. The method may be broadly defined as including the steps of:

(1) treating a skin area with an alpha hydroxy acid to exfoliate that skin area;

(2) providing a patch containing the selected drug and a vehicle for enhancing the transdermal delivery of that drug; and

(3) applying that patch to the treated skin area.

In the most preferred embodiment that alpha hydroxy acid utilized is selected from a group of compounds consisting of glycolic acid, lactic acid, citric acid and any mixtures thereof. Further, the vehicle used is propylene glycol although other known vehicles could be utilized. Still further, the method also preferably includes the step of cleaning the skin area treated with the alpha hydroxy acid in order to remove any alpha hydroxy acid residue and aid in the transdermal delivery of the selected drug. Preferably, normal saline is utilized when cleaning.

Advantageously, the present method provides a convenient and essentially painless approach for the safe transdermal delivery of a wide variety of drugs and immunizations that may be effectively utilized to treat adults and children alike the world over.

Still other objects of the present invention will become apparent to those skilled in this art from the following description wherein there is shown and described a preferred embodiment of this invention, simply by way of illustration of one of the modes best suited to carry out the invention. As it will be realized, the invention is capable of other different embodiments and its several details are capable of modification in various, obvious aspects all without departing from the invention. Accordingly, the drawings and descriptions will be regarded as illustrative in nature and not as restrictive.

### BRIEF DESCRIPTION OF THE DRAWING

The accompanying drawing incorporated in and forming a part of the specification, illustrates several aspects of the present invention and together with the description serves to explain the principles of the invention. In the drawing:

FIG. 1 is a schematical block diagram in side elevation illustrating a transdermal delivery patch of the type utilized in the present method; and

FIG. 2 is a bottom plan view of the patch shown in FIG. 1 with the protective liner removed.

Reference will now be made in detail to the present preferred embodiment of the invention, an example of which is illustrated in the accompanying drawing.

### DETAILED DESCRIPTION OF THE INVENTION

As briefly described above, the present method broadly includes the step of preparing an area of the skin by using an alpha hydroxy class of acid (e.g. glycolic acid, lactic acid, citric acid and mixtures thereof) to exfoliate the skin area. Specifically, the skin preparation is carried out by rubbing the skin with a swab or patch treated with the alpha hydroxy acid (preferably having a strength of between 12-25% by titration) so as to enhance permeation of the dermis. This has the effect of gently exfoliating dead cells from the stratum corneum epidermis layer of the skin. Advantageously, this allows the applied medication to be in close, prolonged contact with the dermis which has rich vascularity, thus promoting microcirculational contact and more efficient and effective drug administration.

More particularly, one of the difficulties associated with transdermal drug delivery is that the penetration of a drug into the viable epidermis and dermis may be difficult to achieve. The stratum corneum serves as a reservoir phase or depot wherein topically applied drug is absorbed and accumulates. This can limit the subsequent penetration of the applied drug. In fact, the greatest resistance to penetration is met in the stratum corneum. Advantageously, the present method of transdermal drug delivery enhances drug penetration by means of increasing pore size resulting from the alpha hydroxy acid skin preparation.

Next, the method includes the step of cleansing by rubbing or rinsing the treated area of the skin with normal saline. This serves to eliminate substantially any alpha hydroxy acid residue and prevent any possibility of irritation. Further, the normal saline is isotonic and helps to transport the selected drug through the skin so as to further enhance transdermal delivery.

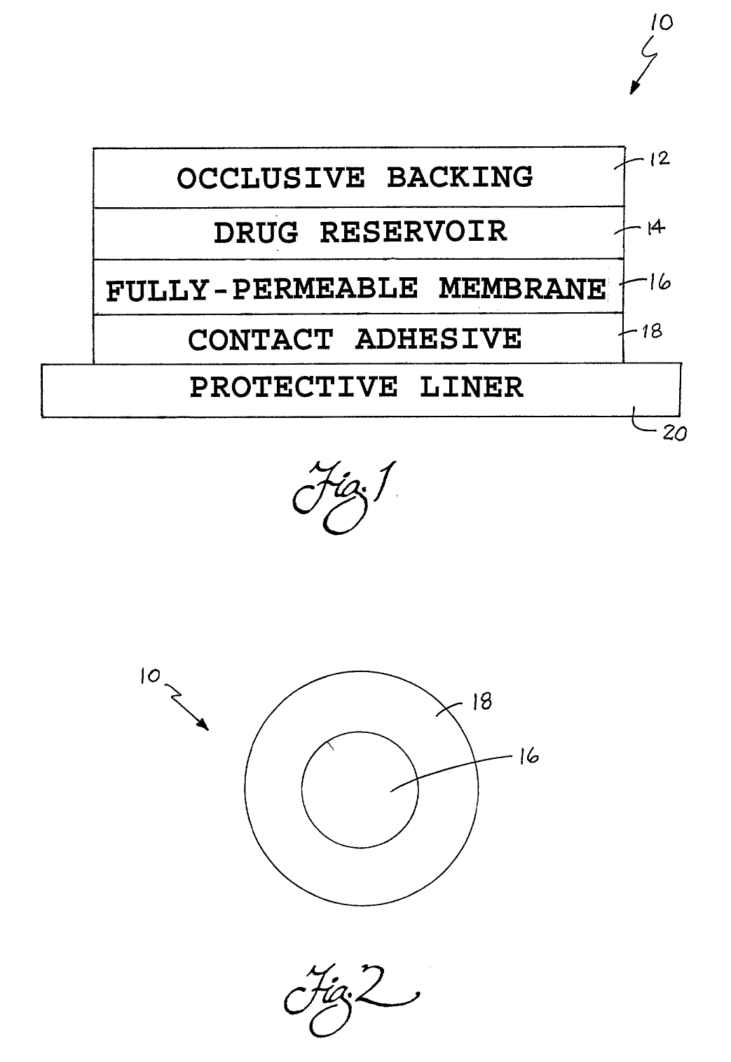

Next, the method includes the step of providing a patch containing the selected drug which is to be delivered. As shown in FIGS. 1 and 2, the patch 10 includes an occlusive backing 12, a drug reservoir 14, a fully permeable membrane 16, a contact adhesive 18 and a protective liner 20. The patch is prepared so as to contain propylene glycol in the drug reservoir 14. This propylene glycol functions as a vehicle for enhancing the transdermal delivery of the drug being administered. The propylene glycol acts as a solvent to polarize the drug or vaccine molecule for transcutaneous passage through the lipid bilayer membrane of the skin. The bicontinuous intercellular pathway then preferentially permeates and networks in the nonpolar and polar diffusion pathway between the corneocytes.

Next, the method includes the step of applying the patch 10 to the skin. Specifically, the protective liner 20 is removed and the contact adhesive 18 is exposed, allowing the patch 10 to be adhered to the skin previously treated or prepared with the glycolic acid. Advantageously, this allows the drug to be effectively and painlessly adsorbed though the skin in a far more efficient and effective manner than heretofore achieved in the art.

One of the greatest benefits of the present invention is its versatility as an immunization/drug delivery system. The selected drug which may be administered by using the present method may include but is not limited to drugs for immunizing or vaccinating against, for example, DPT, MMR, hepatitis B, polio and chicken pox. Further, the present method may also be utilized to administer insulin to diabetics requiring insulin.

The foregoing description of a preferred embodiment of the invention has been presented for purposes of illustration and description. It is not intended to be exhaustive or to limit the invention to the precise form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled.

1. A method for the transdermal delivery of a selected drug, comprising the steps of:
treating a skin area with alpha hydroxy acid to exfoliate the skin area;
providing a patch containing the selected drug, the patch also containing a vehicle for enhancing the transdermal delivery of the selected drug; and
applying the patch to the treated skin area.

2. The method set forth in claim 1, wherein said vehicle is propylene glycol.

3. The method set forth in claim 1, wherein said alpha hydroxy acid is selected from a group of compounds consisting of glycolic acid, lactic acid, citric acid and any mixtures thereof.

4. The method set forth in claim 1, further including cleaning said skin area treated with said alpha hydroxy acid in order to remove any alpha hydroxy acid residue and aid in the transdermal delivery of the selected drug.

5. The method set forth in claim 1, further including cleaning said skin area treated with alpha hydroxy acid with normal saline.
